(19) United States
(12) Patent Application Publication
(10) Pub. No.: US 2002/0173043 A1
(43) Pub. Date: Nov. 21, 2002

(54) Cyanide-free reagent, and method for detecting hemoglobin

(76) Inventors: Eddine Merabet, St. Louis, MO (US); Karina Shapiro, Ballwin, MO (US); Melissa Cousino, Creve Coeur, MO (US)

Correspondence Address: Stephen M. Haracz, Esq., Bryan Cave, LLP, 245 Park Avenue, New York, NY, 10167-0034, US

(21) Appl. No.: 09/826,468

(22) Filed: Apr. 4, 2001

(51) Int. Cl. G01N 33/72

(52) U.S. Cl. 436/66; 422/61

(57) ABSTRACT

A cyanide-free reagent for detecting hemoglobin is provided. The cyanide-free reagent includes a surfactant, a cyanide-free ligand selected from a nitrate, a nitrate salt, a nitrite, a nitrite salt, and combinations thereof, and a hydrogen ion concentration sufficient to maintain the pH of the reagent below about 9. A method and a kit for detecting hemoglobin in a blood sample using the cyanide-free reagent are also provided.

### FIELD OF THE INVENTION

The present invention relates to a cyanide-free reagent for detecting hemoglobin. More particularly, the invention relates to a cyanide-free reagent containing a surfactant and a cyanide-free ligand, which are maintained at a pH below about 9. Methods and kits using the cyanide-free reagent for detecting hemoglobin in blood are also provided.

### BACKGROUND OF THE INVENTION

The measurement of hemoglobin concentration in a whole blood sample is useful for clinical diagnosis of diseases such as leukemia, anemia, polycythemia, and other hematological disorders. As is well known, hemoglobin is located in erythrocytes (i.e., red blood cells) and functions to transport oxygen from the lungs to various tissues and organs in the body. The determination of hemoglobin concentration in a patient's blood sample is one of the most common, and important hematological assays ordered in the clinical setting.

Current methods utilize spectrophotometry to quantitate the amount of hemoglobin in a blood sample. These methods typically require that the hemoglobin be released from the erythrocyte, and that the hemoglobin be converted into a single chromogenic species.

The classical method for detecting hemoglobin in a blood sample utilizes the method of Drabkin. (D. L. Drabkin and J. H. Austin, Spectrophotometric Studies, J. BIOL. CHEM., 112:51, 1935). In a modern adaptation of this method, an erythrolytic agent containing a cationic surfactant at a pH above 10 is mixed with a blood specimen to hemolyze the erythrocytes and to release the hemoglobin. Potassium ferricyanide is then added to the mixture, which results in oxidation of the heme iron to produce cyanomethemoglobin. Cyanide ions then convert the methemoglobin to cyanomethemoglobin, a more stable chromagen.

The hemoglobin concentration of the sample is determined by measuring the absorbance of the cyanomethemoglobin at 540 nm. Although this adaptation of Drabkin is able to be used in currently available hematology analyzers, this method is disadvantageous because it requires the use of highly toxic cyanide, and the maintenance of a pH above 10.

Benezra et al., U.S. Pat. No. 4,853,338 (Benezra '338) disclose a cyanide-free reagent that uses hydroxide anions as heme oxidizing and binding ligands, and a surfactant at 2% to 5% (v/v) to lyse cells and release the hemoglobin. This reagent is disadvantageous because it requires that the pH be maintained at 11.3 or above.

Kim et al., U.S. Pat. No. 5,612,223 disclose a cyanide-free reagent for determining hemoglobin concentrations in blood samples The reagent consists of a heme-binding ligand and a surfactant, which are adjusted to a pH of 11 to about 14. Like Benezra '338, this reagent is disadvantageous because it requires that a high pH be maintained.

Typically, hemoglobin determination is only one of a number of diagnostic tests ordered on a patient's blood sample. Many of such tests are run at or close to physiological pH, i.e., below about 9. As set forth in more detail below, however, many current hemoglobin tests require high pH (i.e., between 11-14). Because of this limitation, separate tests must be run. When the amount of blood is in short supply, this may require foregoing certain tests that would have ordinarily been ordered. Moreover, having to run multiple tests on different blood samples is inefficient, and may pose a risk to life when the clinician requires immediate results.

For example, glycated hemoglobin assays provide an index of the mean concentration of blood glucose during the two months preceding the test. Such assays are used to monitor the long-term blood glucose control and compliance in patients with type I and type II diabetes mellitus. Such assays may require measurement of both glycated hemoglobin and total hemoglobin. Many glycated hemoglobin assays require that the pH of the sample be maintained between about 7 and about 8. Thus, such assays cannot be run together with a hemoglobin assay that requires a pH between about 11 and about 14.

In sum, all of the documents summarized above suffer from the disadvantage of having to rely on a toxic heme binding ligand, i.e., cyanide, and/or require a high pH to maintain the stability of the heme molecule. Thus, the cyanide containing reagents and methods summarized above pose health risks to technicians who run the tests, and environmental hazards when the spent reagents are disposed of. Likewise, the reagents and methods summarized above that rely on high pH to lyse the erythrocytes and stabilize the heme are inconvenient to use because they cannot be combined with other blood tests typically ordered by a clinician, which require pHs below 9.

### SUMMARY OF THE INVENTION

Accordingly, it would be desirable to provide a cyanide-free reagent for measuring hemoglobin in a blood sample, which reagent functions at a pH below about 9.

It would also be desirable to provide a cyanide-free reagent that may be used in conjunction with other hematological assays that require a pH below about 9.

It would also be desirable to provide a method and kit for determining hemoglobin using a cyanide-free reagent that functions at a pH below about 9.

It would further be desirable to provide a cyanide-free reagent, a method of using such a reagent, and a kit containing such a reagent, wherein other commonly ordered hematological assays may be performed at the same time, using the same blood sample.

These and other disadvantages of the prior art are overcome by the present invention.

One embodiment of the present invention is a cyanide-free reagent for detecting hemoglobin. This reagent contains a surfactant and a cyanide-free ligand selected from a nitrate, a nitrate salt, a nitrite, a nitrite salt, and combinations thereof, and a hydrogen ion concentration sufficient to maintain the pH of the reagent below about 9.

Another embodiment of the present invention is a method for detecting hemoglobin in a blood sample. This method includes combining a blood sample with a cyanide-free reagent containing a surfactant and a cyanide-free ligand selected from a nitrate, a nitrate salt, a nitrite, a nitrite salt, and combinations thereof, and a hydrogen ion concentration sufficient to maintain the pH of the reagent below about 9. The absorbance of the chromogen formed by reaction of the ligand with the heme in the blood sample is then measured.

A further embodiment of the present invention is a kit for detecting hemoglobin in a blood sample. This kit includes a cyanide-free reagent consisting of a surfactant and a cyanide-free ligand selected from a nitrate, a nitrate salt, a nitrite, a nitrite salt, and combinations thereof, and a hydrogen ion concentration sufficient to maintain the pH of the reagent below about 9.

### BRIEF DESCRIPTION OF THE DRAWINGS

FIG. 1 is a graph comparing the absorbance spectra of a whole blood sample treated with a cyanide-free reagent containing sodium nitrite according to the present invention and a whole blood sample treated with cyanide-free reagent without sodium nitrite.

FIG. 2 is a graph of the absorbance spectra of whole blood samples maintained at various pHs when treated with the cyanide-free reagent according to the present invention.

FIG. 3 is a graph of absorbance vs. time of a whole blood sample treated with a cyanide-free reagent according to the present invention.

FIG. 4 is a correlation plot of hemoglobin data obtained using the cyanide-free reagent of the present invention at pH 7 versus the Oshiro reagent in Example 4.

FIG. 5 is a correlation plot of hemoglobin data obtained using the cyanide-free reagent of the invention at pH 12 versus the Oshiro reagent in Example 4.

FIG. 6 is a correlation plot of hemoglobin data obtained using the cyanide-free reagent of the present invention containing nitrite versus the same reagent using nitrate.

FIG. 7 is a graph showing the stability of hemoglobin in a reagent according to the present invention.

FIG. 8 is a correlation plot of % HbA1c determined using the cyanide-free reagent of the present invention and an HbA1c assay compared to % HbA1c determined using a conventional HbA1c assay.

### DETAILED DESCRIPTION OF THE INVENTION

One embodiment of the present invention is a cyanide-free reagent for detecting hemoglobin. This reagent includes at least one surfactant, and a cyanide-free ligand selected from a nitrate, a nitrate salt, a nitrite, a nitrite salt, and combinations thereof, and a hydrogen ion concentration sufficient to maintain the pH of the reagent below about 9.

In the present invention, nitrites, nitrite salts, nitrates, nitrate salts, and combinations thereof not only completely oxidize hemoglobin to methemoglobin but also stabilize it for a period long enough to accommodate the automated analyzers commonly used to determine hemoglobin concentrations in blood sample.

In the present invention, the cation counterpart to the cyanide-free ligand may be sodium, potassium, magnesium, amyl, butyl, or any cation capable of forming a cyanide-free salt with nitrite or nitrate. Preferably, the cation is sodium. The cyanide-free ligand is preferably either sodium nitrite or sodium nitrate. In the present invention, “cyanide-free” is used to indicate that the heme-binding ligand, as well as the reagent itself are free of cyanide.

In the present invention, the cyanide-free ligand is present in the cyanide-free reagent at a concentration from about 0.05 M to about 2 M. Preferably, the cyanide-free ligand is present in the cyanide-free reagent at a concentration of about 0.5 M to about 1.5 M, such as for example, about 1 M.

The cyanide-free reagent of the present invention also includes a surfactant with a strong erythrolytic capability. As used herein, “a strong erythrolytic capability” means that the selected surfactant is able to lyse all or at least 95%, preferably 98%-100%, of the erythrocytes in a blood sample combined with the cyanide-free reagent of the present invention.

Thus, in the present invention, the surfactant may be selected from the group of β-mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, or mixtures thereof. Preferably, the surfactant is octyl phenoxypolyoxyethanol (Triton X-100).

In the present invention, the surfactant is present in the cyanide-free reagent at concentrations sufficient to lyse all or at least 95%, preferably 98%-100%, of the erythrocytes in a blood sample to be analyzed. Typically, the surfactant is present in the cyanide-free reagent at a concentration ranging from about 0.1% to about 3% (v/v) Preferably, the surfactant is present in the cyanide-free reagent at about 0.5% to about 2% (v/v), such as for example, at about 1% (v/v).

Preferably, the cyanide-free reagent contains sodium nitrite and octyl phenoxyethoxyethanol at a pH of 7.4. Other optional reagents well known in the art may be combined with the cyanide-free reagent. For example, preservatives may be added to the cyanide-free reagent to keep the reagent free of bacteria. Sodium azide is one example of a preservative that may be added.

As noted above, the cyanide-free reagent according to the present invention is maintained at a pH below about 9. In other words, the reagent contains a hydrogen ion concentration sufficient to maintain the pH of the reagent below about 9. Preferably, the pH of the cyanide-free reagent is less than about 8, such as for example, between about 7 to about 8. In another preferred embodiment, the pH of the reagent is about 7 4. As used herein, “a pH below about 9,” “a pH . . . of less than about 8″ and a “pH of between about 7 to about 8″ are intended to indicate that the hydrogen ion concentration is maintained at, or adjusted to the specified pH. Adjustments in the hydrogen ion concentration (pH) are well within the skill of the art, and are typically achieved using an acid or a base as necessary.

In the present invention, it is convenient to select the surfactant and the cyanide-free ligand so that the pH of the cyanide-free reagent is maintained below about 9, preferably between 7 and 8, such as at about 7.4. Thus, no adjustment of the pH is required by, e.g., an acid or base.

By maintaining the pH of the reagent below about 9, the cyanide-free reagent of the present invention may be combined with one or more hematological assays that are commonly ordered by a clinician, which typically require a pH of below about 9. Examples of typically ordered hematological assays include the following in Interpretive Data For Diagnostic Laboratory Tests, 264-68 (Mayo Press 1997):

Glycohemoglobin tests (Hemoglobin A1 or A1c, HbA1c): Glycohemoglobin measures the amount of glucose chemically attached to a patient's red blood cells. And, as noted above, is a monitor for long-term blood glucose control and compliance with patients with type I and II diabetes mellitus.

Hemoglobin A₂ tests Hemoglobin A₂ is a hemoglobin variant normally found in blood. Elevated levels of hemoglobin A₂ are characteristic of the genetic disorder beta-thalassemia. Additionally, a slight elevation in hemoglobin A₂ may also indicate a vitamin B₁₂ or folate deficiency or hyperthyroidism.

Hemoglobin electrophoresis tests Hemoglobin contains numerous variants the presence, absence, or levels of which may be clinically significant. Hemoglobin electrophoresis uses high-performance liquid chromatography (HPLC) to identify and measure the hemoglobin variants found in a blood sample.

Fetal Hemoglobin (Hemoglobin F) tests Low levels of hemoglobin F are normally found in adult blood (0-2%; up to 5% during a normal pregnancy.) Elevated hemoglobin F levels may indicate various disorders including: beta-thalassemia, delta-thalassemia, aplastic anemia, hereditary spherocytosis, myeloproliferative disorders, sickle cell disease, and S/beta O-thalassemia. Further, patients who are doubly heterozygous for the hemoglobin S gene or a gene for hereditary persistence of fetal hemoglobin will exhibit elevated hemoglobin F levels.

Plasma hemoglobin tests Hemoglobin is normally not found in the plasma of a healthy patient Accordingly, the presence of hemoglobin in plasma may indicate the occurrence of a significant hemolytic event Such hemolytic events may include transfusion reactions and mechanical fragmentation of red blood cells during cardiac surgery.

Hemoglobin S tests The presence of hemoglobin S is used to screen for homozygous hemoglobin S disease. Homozygous hemoglobin S disease is a serious chronic hemolytic anemia. Hemoglobin S is freely soluble when fully oxygenated, and when deoxygenated polymerization of the hemoglobin occurs forming tactoids that are rigid and deformed cells.

Unstable hemoglobin tests Unstable hemoglobins are easily denatured, and their presence in the blood may indicate hemolytic anemia

Such assays, may be combined with the present cyanide-free reagent so that hemoglobin determination may be accomplished at the same time, and with the same blood, as one or more of the above assays. The hematological assays set forth herein are intended to be illustrative of the types of assays that may be combined with the present cyanide-free reagent. Other hematological assays that require maintaining a blood sample at a pH below about 9 may also be used in combination with the present cyanide-free reagent.

Typically, the present cyanide-free reagent is added to the blood sample first lyse the and stabilize the heme. Thereafter reagent(s) for one or more of the previously identified assays is/are added to the blood sample. Then the hemoglobin and other hematological parameter(s) are measured.

Another embodiment of the present invention is a method for detecting hemoglobin in a blood sample. As used herein, “blood” and “whole blood” are used interchangeably, and both refer to a blood sample containing erythrocytes, i.e., red blood cells.

In this method, a blood sample is combined with the cyanide-free reagent defined above. As used herein, “combining” means that the blood sample is mixed with the reagent so that complete lysing of the erythrocytes and oxidation of the heme in the erythrocytes is achieved within a short period of time, preferably within less than a minute, preferably less than 30 seconds, such as for example, less than 10 seconds. The mixing may be carried out manually, or using any well known automated mixing device, so that complete lysing and oxidation of the heme is achieved. As used herein, “complete oxidation of the heme” means that greater than 95% of the heme is oxidized, preferably greater than 98% of the heme is oxidized, such as 100% of the heme is oxidized.

Upon combining the blood sample with the cyanide-free reagent of the present invention, the mixture immediately turns to a dark-green color indicating that red blood cell lysing and heme oxidation and ligation is complete. The absorbance, i.e., optical density, of the chromogen formed by the reaction of the cyanide-free reagent and the heme in the blood is then read using a spectrophotometer capable of reading absorbances at between about 400 nm and about 700 nm. Preferably, the spectrophotometer is a Beckman DU-7 Spectrophotometer (Beckman Instruments, Fullerton, Calif.).

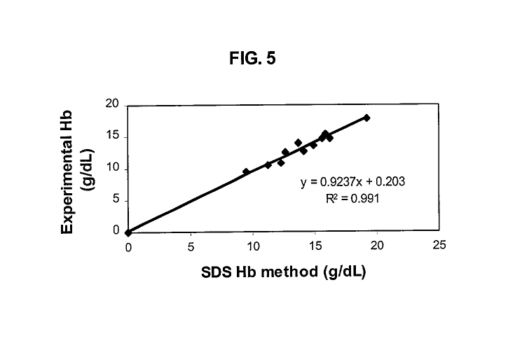

The wavelength at which the optical density (absorbance) of the chromogen is measured depends on the pH of the mixture. For a pH below about 9, peaks appear at wavelengths of about 540 nm and about 570 nm. For a pH above about 9, peaks are observed at wavelength of about 570 nm and about 600 nm. Preferably, the absorbance is read at about 540 nm or about 570 nm. Although the present cyanide-free reagent is capable of lysing erythrocytes and oxidizing and ligating heme at pHs above 9 (see FIG. 5), a benefit of the present reagent is that it is just as effective at pHs below about 9. And, therefore, may be combined with a variety of other hematological assays typically ordered by a clinician.

In the present method, the cyanide-free ligand is preferably sodium nitrite or sodium nitrate. The surfactant in this method may be selected from the group of β-mercaptoethanol, mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, or mixtures thereof. Preferably, the surfactant is octyl phenoxypolyoxyethanol (Triton X-100).

In this method, the pH of the cyanide-free reagent is adjusted to less than about 8, preferably about 7.4. More preferably, the cyanide-free ligand and surfactant are selected so that the pH of the cyanide-free reagent does not have to be adjusted at all, and is maintained at about 7.4. If necessary, the pH may be adjusted upward or downward, as needed, using a weak acid or base.

Preferably, the cyanide-free reagent in this method contains sodium nitrite and octyl phenoxypolyethoxyethanol at a pH of about 7.4.

Another embodiment of the invention is a kit for detecting hemoglobin in a blood sample. The kit includes the cyanide-free reagent of the present invention. The kit is packaged so that the cyanide-free reagent is provided in a single, multi-use container. Typically, the container will hold about 100 ml to about 1 L of the cyanide-free reagent. Containers of less or greater volume may also be selected based on the end-user requirements.

The container may be designed to specifically fit into a commercially available high-throughput clinical analyzing device. Alternatively, the container may be designed to accommodate easy pouring of the cyanide-free reagent from the kit's container to another container as required by the end-user.

The cyanide-free reagent in the kit may be provided as a concentrate, such as for example, in a 5×, 10×, 100×, or 1000× concentration. When the cyanide-free reagent is provided as a concentrate, it may be combined with water or an appropriate buffer and diluted to the required concentration. Alternatively, the cyanide-free reagent may be provided as a 1× solution which is ready to be combined with a blood sample.

When it is desired to combine the cyanide-free reagent with one or more hematological assays, the cyanide-free reagent is combined with the blood sample first. In this way, the erythrocytes in the blood sample are lysed, and the heme is oxidized, ligated, and stabilized. Thereafter, the additional reagent(s) for accomplishing one or more additional hematological assays may be added, followed by detection using, e.g., a spectrophotometer.

In the kit according to the present invention, it is preferred that the cyanide-free ligand is sodium nitrite or sodium nitrate.

In the kit according to the present invention, the surfactant may be selected from the group of β-mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, or mixtures thereof Preferably, the surfactant is octyl phenoxypolyoxyethanol (Triton X-100).

Preferably, the cyanide-free reagent used in the kit according to the present invention contains sodium nitrite and octyl phenoxyethoxyethanol at a pH of about 7.4.

The following examples are provided to further illustrate the process of the present invention. These examples are illustrative only and are not intended to limit the scope of the invention in any way.

### EXAMPLES

### Example 1

### Preparation of the Cyanide-Free Reagent

Approximately 700 mL of deionized water was collected in a container. With constant mixing, 1M (69 g) of sodium nitrite was added to the water. Mixing continued until dissolution was complete Triton X-100 (octyl phenoxypolyoxyethanol) was then added to the mix at a concentration of 1% v/v (1 ml/L) Once the Triton X-100 (Sigma, St Louis, Mo.) was completely dissolved, the volume of the solution was adjusted to 1 L with deionized water and filtered through a 0.2 mm filter into a clean container The pH of this solution was measured at 7.4

### Example 2

### Detection of Hemoglobin Using the Cyanide-Free Reagent

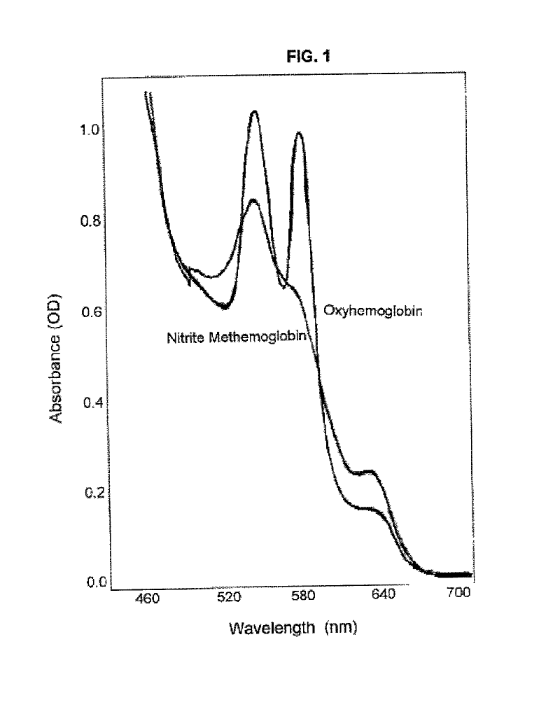

An aliquot (10 uL) of a whole blood sample from a human was added to 2 5 mL of the reagent of Example 1 The absorbance of the chromogen formed was scanned from 700 nm to 400 nm on a Beckman DU-7 spectrophotometer (Beckman Instruments, Fullerton, Calif.) (see FIG. 1, nitrite methemoglobin). As a control, a whole blood sample from a human was mixed with a reagent prepared in the same manner as in Example 1 except that sodium nitrite was omitted, and a scan performed as set forth above (FIG. 1, oxyhemoglobin). FIG. 1 shows that the cyanide-free reagent of the present invention (curve labeled “nitrite methemoglobin”) converted oxyhemoglobin to methemoglobin. The reagent absent the sodium nitrite was unable to oxidize the heme.

### Example 3

### Stability of Cyanide-Free Reagent at Various pHs

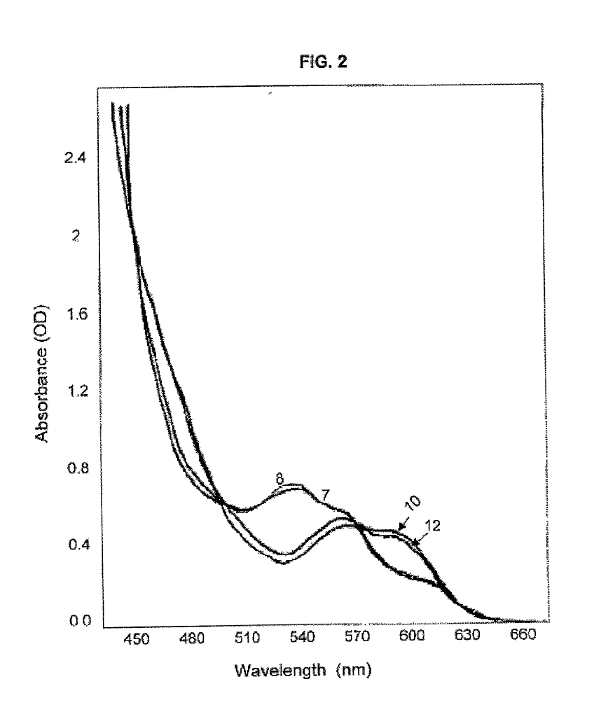

The absorbance curve for methemoglobin is markedly influenced by changes in pH. Cyanide-free reagents were prepared in the same manner as in Example 1 except that the pH was adjusted to 8, 9, 10, and 12 with 0.1 N sodium hydroxide. 2.5 ml of each reagent was then combined with 10 μl of a human blood sample, and its absorbance determined as set forth in Example 2 FIG. 2 is a spectra of each sample As FIG. 2 shows, at lower pH values, the absorbance peaks were at approximately 540 nm and 570 nm. At higher pH values, the absorbance peaks were at approximately 570 nm and 600 nm. Thus, the cyanide-free reagent is stable across a range of pHs from about 7.4 (Example 1) to at least about 12.

### Example 4

### Chromogen Formation and Stability

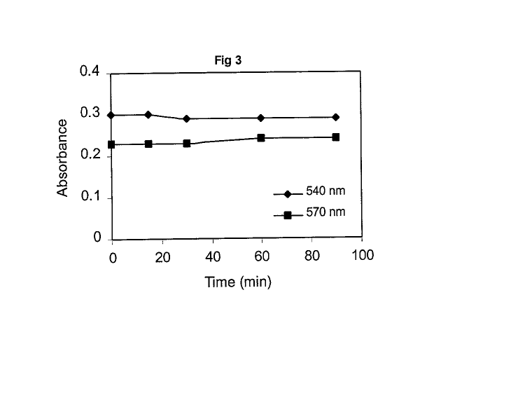

A whole blood sample (10 μL) from a human was mixed with 2.5 mL of the cyanide-free reagent prepared according to Example 1 The absorbance of the mixture was monitored with time to evaluate the completeness and stability of the formation of chromogen. FIG. 3 shows the absorbance of the mixture at wavelengths of 540 nm and 570 nm at various times after the reagent and the blood were mixed As shown in FIG. 3, the lysis of the erythrocytes and conversion (of hemoglobin to methemoglobin) was complete within 10 seconds and the chromogen remained stable for at least 90 minutes after mixing.

### Example 5

The performance of two different formulations (i.e., formulation 1 and 2, set forth below) of the cyanide-free reagent of the present invention were compared to a hemoglobin measurement method published by Oshiro et al. in CLINICAL BIOCHEMISTRY, vol. 15, 83 (1982). The Oshiro reagent contained an anionic surfactant, sodium dodecyl sulfate (SDS) or sodium lauryl sulfate (SLS), and a nonionic surfactant such as Triton X-100.

Formulation 1:

1M sodium Nitrite (69 g/L)

Triton X-100 (1 mL/L)

pH7.4

Formulation 2:

1M sodium Nitrite (69 g/L)

Triton X-100 (1 mL/L)

pH 12 (adjusted with 1 M NaOH)

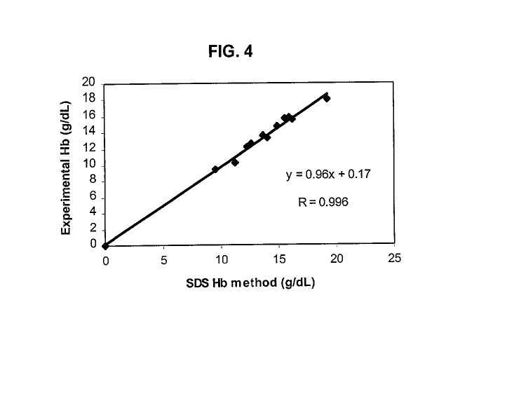

Reagent samples for formulations 1 and 2 were prepared in the same manner as in Example 2 2.5 ml of the Oshiro reagent and formulations 1 and 2 were mixed with 10 μl of human whole blood. Table 1 represents results obtained with 12 whole blood samples analyzed on a Beckman DU-7 Spectrophotometer (Beckman Instruments, Fullerton, Calif.) All three assays were calibrated with a whole blood sample assayed with Drabkin's method. FIGS. 4 (Oshiro vs. Formulation 1) and 5 (Oshiro vs. Formulation 2) are correlation plots showing the correlation between the data in the columns labeled “Formulation 1” and “Formulation 2” and the Oshiro data.

TABLE 1

| Sample | Oshiro method | Formulation 1 | Formulation 2 |
|---|---|---|---|
| 1 | 0 | 0 | 0 |
| 2 | 12.6 | 12.6 | 12.6 |
| 3 | 9.5 | 9.5 | 9.3 |
| 4 | 11.2 | 10.3 | 10.5 |
| 5 | 12.3 | 12.2 | 10.7 |
| 6 | 141 | 13.4 | 127 |
| 7 | 13.7 | 13.6 | 13 9 |
| 8 | 15.0 | 14.7 | 13.6 |
| 9 | 15.6 | 15.7 | 14.7 |
| 10 | 15.9 | 15.8 | 15.5 |
| 11 | 192 | 18.0 | 17.8 |
| 12 | 16.2 | 15.5 | 14.6 |

### Example 6

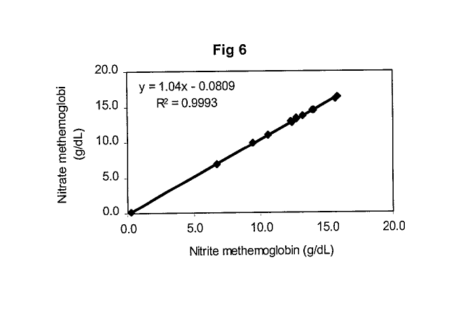

A reagent sample containing sodium nitrite and a reagent sample containing sodium nitrate were prepared as in Example 1. FIG. 6 shows the correlation of the hemoglobin concentrations as measured using the nitrite reagent versus the nitrate reagent. The figure demonstrates that the results are virtually identical as indicated by linear regression analysis. (slope=1.04, intercept=0.0809, and r=0.9993.)

### Example 7

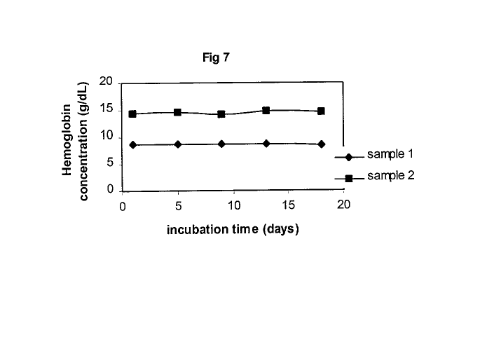

The cyanide-free reagent was prepared according to Example 1. FIG. 7 shows the hemoglobin concentration as measured at various times after preparation of the reagent The reagent was stored at 37° C. Two different samples (Sample 1 and Sample 2) of human whole blood were used. Aliquots of each sample of blood were stored until use at −70° C. At the appropriate time after preparation, an aliquot (10 μl) of blood from each sample was separately mixed with the reagent (2.5 ml). The hemoglobin concentration was measured as discussed above for each blood sample.

FIG. 7 shows that the reagent remained stable for at least 18 days at 37° C. From this data the Arrhenius kinetic model was employed to predict that the reagent has a shelf-life of over 2 years. (See J. R. Giacin et al., Predicting Packaged Product Shelf Life: Experimental and Mathematical Model, PHARMACEUTICAL TECHNOLOGY, pg. 98-116, September 1991; and T. B. L Kirkwood & M. S Tydeman, Design and Analysis of Accelerated Degradation Tests for the Stability of Biological Standards II. A Flexible Computer Program for Data Analysis, J BIOL STANDARDIZATION, 12: 207-214, 1984.)

### Example 8

### Glycated Hemoglobin Percentage Assay

The cyanide-free reagent was prepared according to Example 1. Whole blood samples from 50 patients were obtained from a hospital. The HbA1c value for each blood sample was determined by a reference method (Variant II, BioRad Inc.) by the hospital. The blood samples were stored at 2-8° C. and analyzed within 2 to 3 days after collection. A 96-well microtiter plate (Sigma Z37, 182-3) was coated with 0.1 ml/well of an anti-HbA1c antibody reagent (0.05 mg/ml). The plate was then incubated for 30 minutes at 37° C., and then washed three times using approximately 0.3 ml/well of a wash buffer (0.1% Tween, 0.1% BSA in PBS). The plate was then incubated with 0.3 ml/well of a blocking buffer (0.2% BSA in PBS) for 15 minutes at 37° C., and washed three times using approximately 0.3 ml/well of wash buffer. The blood samples were diluted 1:10,000 in the cyanide-free reagent f of Example 1. The total hemoglobin concentration for each blood sample was measured at 540 nm as set forth in Example 2. 0.1 ml/well of the diluted samples was added to a 96-well microtiter plate, and incubated for 30 minutes at 37° C.

The plate was then washed three times using approximately 0.3 ml/well of wash buffer. 0.1 ml/well of the HbA1c reagent was added to the 96-well plate, and incubated for 15 minutes at 37° C. with shaking. The HbA1c reagent is a 1:500 dilution of a rabbit anti-human hemoglobin antibody conjugated to alkaline phosphatase. The plate was washed three times using approximately 0.3 ml/well of wash buffer. 0.1 ml/well of the substrate reagent was added to the 96-well plate, and incubated for 10-20 minutes at 18-25° C. 0.1 ml/well of the stop reagent was added to the 96-well plate. The absorbance was then measured at 550 nm for each blood sample to determine the concentration of glycated hemoglobin.

The percent of the hemoglobin which was glycated (% HbA1c) was calculated as follows:

% HbA1c=(HbA1c signal/total hemoglobin)×100

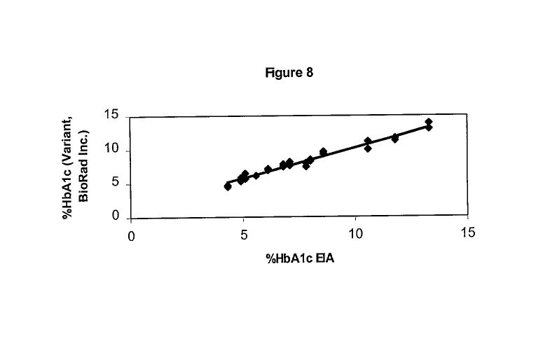

This measured % Hb1Ac was then correlated to the known values for the blood samples (as determined by the hospital using the Variant II process), and reported in FIG. 8. The figure demonstrates that the results obtained using the cyanide-free reagent of the present invention are virtually identical to a commercially available method, as indicated by linear regression analysis. (slope=0 9, intercept=1 2, and r=0 98).

The invention being thus described, it will be obvious that the same may be varied in many ways. Such variations are not to be regarded as a departure from the spirit and scope of the invention and all such modifications are intended to be included within the scope of the following claims.

What is claimed is:

1. A cyanide-free reagent for detecting hemoglobin comprising:
a) a surfactant;
b) a cyanide-free ligand selected from the group consisting of a nitrate, a nitrate salt, a nitrite, a nitrite salt, and combinations thereof; and
c) a hydrogen ion concentration sufficient to maintain the pH of the reagent below about 9

2. A cyanide-free reagent according to claim 1 wherein the cyanide-free ligand is sodium nitrite

3. A cyanide-free reagent according to claim 1 wherein the cyanide-free ligand is sodium nitrate.

4. A cyanide-free reagent according to claim 1 wherein the surfactant is selected from the group consisting of β-mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, and mixtures thereof.

5. A cyanide-free reagent according to claim 4 wherein the surfactant is octyl phenoxypolyethoxyethanol.

6. A cyanide-free reagent according to claim 1 wherein the hydrogen ion concentration is sufficient to maintain the pH of the reagent at less than about 8.

7. A cyanide-free reagent according to claim 1 wherein the hydrogen ion concentration is sufficient to maintain the pH of the reagent at about 7.4.

8. A cyanide-free reagent according to claim 1 wherein the cyanide-free ligand is sodium nitrite, the surfactant is octyl phenoxypolyethoxyethanol, and the pH of the reagent is about 7.4

9. A cyanide-free reagent according to claim 1 wherein the cyanide-free ligand is present in the reagent at about 0.05 M to about 2 M

10. A cyanide-free reagent according to claim 9 wherein the cyanide-free ligand is present in the reagent at about 0.5 M to about 1.5 M

11. A cyanide-free reagent according to claim 10 wherein the cyanide-free ligand is present in the reagent at about 1.0 M.

12. A cyanide-free reagent according to claim 1 wherein the surfactant is present in the reagent at about 0.1% to about 3% (w/v).

13. A cyanide-free reagent according to claim 12 wherein the surfactant is present in the reagent at about 0.5% to about 2% (w/v).

14. A cyanide-free reagent according to claim 13 wherein the surfactant is present in the reagent at about 1% (w/v)

15. A method for detecting hemoglobin in a blood sample comprising the steps of:
a) combining a blood sample with a cyanide-free reagent comprising:
i) a surfactant,
ii) a cyanide-free ligand selected from the group consisting of a nitrate, a nitrate salt, a nitrite, a nitrite salt, and combinations thereof, and
ii) a hydrogen ion concentration sufficient to maintain the pH of the reagent below about 9; and
b) measuring the absorbance of a chromogen formed by reaction of the ligand with the heme in the blood sample.

16. A method according to claim 15 wherein the absorbance is measured at about 540 nm or about 570 nm.

17. A method according to claim 15 wherein the cyanide-free ligand is sodium nitrite or sodium nitrate.

18. A method according to claim 15 wherein the surfactant is a selected from the group consisting of β-mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, and mixtures thereof.

19. A method according to claim 18 wherein the surfactant is octyl phenoxypolyethoxyethanol

20. A method according to claim 15 further comprising adjusting the hydrogen ion concentration of the reagent to a pH of less than about 8.

21. A method according to claim 20 further comprising adjusting the hydrogen ion concentration of the reagent to a pH to about 7.4.

22. A method according to claim 15 wherein the cyanide-free ligand is sodium nitrite, the surfactant is octyl phenoxypolyethoxyethanol, and the pH of the reagent is about 7.4.

23. A kit for detecting hemoglobin in a blood sample comprising the component parts of:
a) a cyanide-free reagent consisting of:
a) a cyanide-free reagent consisting of:
i) a surfactant,
ii) a cyanide-free ligand selected from the group consisting of a nitrate, a nitrate salt, a nitrite, a nitrite salt, and combinations thereof, and
iii) a hydrogen ion concentration sufficient to maintain the pH of the reagent below about 9.

24. A kit according to claim 23 wherein the cyanide-free ligand is sodium nitrite or sodium nitrate.

25. A kit according to claim 23 wherein the surfactant is selected from the group consisting of β-mercaptoethanol, guanidine thiocynate, lauryl dimethylamine oxide, sodium lauryl sulfate, cetyl tri-methyl ammonium bromide, sodium dodecylsulfate, sodium deoxycholate, saponin, octyl phenoxypolyethoxyethanol, sodium deoxycholate, N-lauroylsarcosine, and mixtures thereof.

26. A kit according to claim 23 wherein the surfactant is octyl phenoxypolyethoxyethanol

27. A kit according to claim 23 wherein the cyanide-free ligand is sodium nitrite, the surfactant is octyl phenoxypolyethoxyethanol, and the pH of the reagent is about 7.4.

28. A kit according to claim 23 further comprising an additional reagent for a hematological assay selected from the group consisting of a glycohemoglobin test, a hemoglobin A₂ test, a hemoglobin electrophoresis test, a fetal hemoglobin test, a plasma hemoglobin test, a hemoglobin S test, an unstable hemoglobin test, and combinations thereof.
